(19) United States
(12) Patent Application Publication
(10) Pub. No.: US 2005/0282709 A1
(43) Pub. Date: Dec. 22, 2005

(54) Substituted benzoylpyrazoles as herbicides

(75) Inventors: Andreas van Almsick, Karben (DE); Lothar Willms, Hofheim (DE); Thomas Auler, Bad Soden (DE); Christopher Rosinger, Hofheim (DE); Heinz Kehne, Hofheim (DE); Martin Hills, Idstein (DE); Dieter Feucht, Eschborn (DE)

Correspondence Address: CONNOLLY BOVE LODGE & HUTZ, LLP, P O BOX 2207, WILMINGTON, DE, 19899, US

(73) Assignee: Bayer CropScience GmbH, Frankfurt am Main (DE)

(21) Appl. No.: 11/156,314

(22) Filed: Jun. 17, 2005

(30) Foreign Application Priority Data
Jun. 17, 2004 (DE) 10 2004 029 308.2

(51) Int. Cl. A01N 43/56; C07D 45/02

(52) U.S. Cl. 504/280; 548/365.7

(57) ABSTRACT

A description is given of benzoylpyrazoles of the formula (I) and of their use as herbicides. 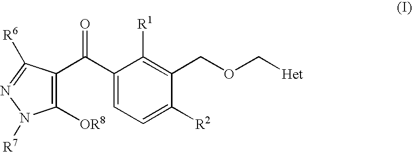
In this formula (I) R¹, R², R⁶, R⁷ and R⁸ are different radicals and Het is a saturated heterocyclic group including oxygen atoms and carbon atoms.

The invention pertains to the technical field of herbicides, particularly that of herbicides from the class of the benzoylpyrazoles for selectively controlling broadleaf and gramineous weeds in crops of useful plants, especially in rice crops.

From a variety of publications it is already known that certain benzoyl derivatives possess herbicidal properties. For instance, WO 99/10327 and WO 99/10328 disclose benzoylcyclohexanediones and benzoylpyrazolones which in position 3 of the phenyl ring carry a heterocyclyl or heteroaryl radical attached via a polyatomic bridge. In German patent application DE 103 01 110.2, which has an earlier priority date but was unpublished at the priority date of the present specification, describes benzoylpyrazolones which in position 3 of the phenyl ring carry a heterocyclyl radical attached via an oxymethyl or thiomethyl bridge.

The compounds known from these publications, however, frequently exhibit an inadequate herbicidal activity.

It is an object of the present invention to provide further herbicidally active compounds having improved herbicidal properties—improved, that is, over those of the prior art compounds.

It has now been found that benzoylpyrazoles which in position 3 of the phenyl ring carry a heterocyclyl radical attached via a triatomic bridge are especially suitable for use as herbicides. The present invention accordingly first provides compounds of the formula (I) or salts thereof 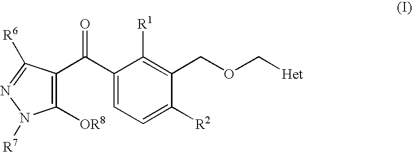
in which the radicals and indices have the following definitions:
R¹ and R² independently of one another are hydrogen, mercapto, nitro, halogen, cyano, thiocyanato, (C₁-C₆)-alkyl, (C₁-C₆)-haloalkyl, (C₂-C₆)-alkenyl, (C₂-C₆)-haloalkenyl, (C₂-C₆)-alkynyl, (C₃-C₆)-haloalkynyl, (C₃-C₆)-cycloalkyl, OR⁴, OCOR⁴, OSO₂R⁴, S(O)ₙR⁴, SO₂OR⁴, SO₂N(R⁴)₂, NR⁴SO₂R⁴, NR⁴COR⁴, (C₁-C₆)-alkyl-S(O)ₙR⁴, (C₁-C₆)-alkyl-OR⁴, (C₁-C₆)-alkyl-OCOR⁴, (C₁-C₆)-alkyl-OSO₂R⁴, (C₁-C₆)-alkyl-SO₂R⁴, (C₁-C₆)-alkyl-SO₂N(R⁴)₂ or (C₁-C₆)-alkyl-NR⁴COR⁴;
R³ is hydrogen, (C₁-C₆)-alkyl, (C₂-C₆)-alkenyl or (C₂-C₆)-alkynyl;
R⁴ is hydrogen,(C₁-C₆)-alkyl, (C₂-C₆)-alkenyl, (C₂-C₆)-alkynyl, (C₃-C₆)-cycloalkyl, phenyl or phenyl-(C₁-C₆)-alkyl, the six last-mentioned radicals being substituted by s radicals from the group consisting of hydroxyl, mercapto, amino, cyano, nitro, thiocyanato, OR³, SR³, N(R³)₂, NOR³, OCOR³, SCOR³, NR³COR³, CO₂R³, COSR³, CON(R³)₂, (C₁-C₄)-alkyliminooxy, (C₁-C₄)-alkoxyamino, (C₁-C₄)-alkylcarbonyl, (C₁-C₄)-alkoxy-(C₂-C₆)-alkoxycarbonyl and (C₁-C₄)-alkylsulfonyl;
R⁶ and R⁷ independently of one another are hydrogen, (C₁-C₆)-alkyl, (C₁-C₆)-haloalkyl or (C₃-C₆)-cyclopropyl;
R⁸ is hydrogen, (C₁-C₆)-alkyl, (C₁-C₆)-haloalkyl, (C₁-C₆)-alkylcarbonyl, (C₁-C₆)-haloalkylcarbonyl, (C₁-C₆)-alkoxycarbonyl, (C₁-C₆)-alkylsulfonyl, (C₁-C₆)-haloalkylsulfonyl, phenylcarbonyl, phenylcarbonylmethyl, phenyloxycarbonyl or phenylsulfonyl, the phenyl ring of the four last-mentioned radicals being substituted by s radicals from the group consisting of halogen, nitro, cyano, (C₁-C₆)-alkyl, (C₁-C₆)-haloalkyl, (C₁-C₆)-alkoxy and (C₁-C₆)-haloalkoxy;
Het is a fully saturated heterocyclic group whose ring atoms are composed of 2 oxygen atoms and 2, 3, 4 or 5 carbon atoms;
and Het is substituted by n radicals R⁵;
n is 0, 1 or 2;
s is 0, 1, 2 or 3;
R⁵ is hydroxyl, mercapto, amino, cyano, nitro, halogen, formyl, (C₁-C₆)-alkylamino, (C₁-C₆)-dialkylamino, (C₁-C₆)-alkoxycarbonyl, (C₁-C₆)-alkylcarbonyl, (C₁-C₄)-alkylcarbonyloxy, (C₁-C₆)-alkyl, (C₁-C₆)-haloalkyl, (C₁-C₆)-alkylthio, (C₁-C₆)-haloalkylthio, (C₁-C₆)-alkoxy or (C₁-C₆)-haloalkoxy or R⁵ together with the carbon atom to which it is attached forms a carbonyl group, or two R⁵s together with the carbon atom to which they are attached form a 3- to 6-membered spiro ring.

Depending on external conditions, such as solvent and pH, it is possible for the compounds of the formula (I) according to the invention to occur in different tautomeric structures. Depending on the nature of the substituents the compounds of the formula (I) contain an acidic proton which can be removed by reaction of a base. Examples of suitable bases include hydrides, hydroxides and carbonates of alkali metals and alkaline earth metals, such as lithium, sodium, potassium, magnesium and calcium, and also ammonia and organic amines such as triethylamine and pyridine. Such salts are likewise provided by the invention.

In formula (I) and all formulae below it is possible for alkyl radicals having more than two carbon atoms to be straight-chain or branched. Alkyl radicals are, for example, methyl, ethyl, n-propyl or isopropyl, n-, iso, t- or 2-butyl, pentyls and hexyls, such as n-hexyl, isohexyl and 1,3-dimethylbutyl.

Where a group is substituted multiply by radicals, this is to be understood to mean that that group is substituted by one or more, identical or different radicals from among those stated.

Cycloalkyl is a carbocyclic, saturated ring system having three to nine carbon atoms, e.g., cyclopropyl, cyclopentyl or cyclohexyl. Similarly, cycloalkenyl is a monocyclic alkenyl group having three to nine carbon ring members, e.g., cyclopropenyl, cyclobutenyl, cyclopentenyl and cyclohexenyl, the double bond being in any desired position. In the case of composite radicals, such as cycloalkylalkenyl, the first-mentioned radical may be situated at any position on the second-mentioned.

The heterocyclic group Het comprehends radicals such as 1,3-dioxetan-2-yl, 1,3-dioxolan-2-yl, 1,3-dioxolan4-yl, 1,3-dioxan-2-yl, 1,3-dioxan-4-yl, 1,3-dioxan-5-yl, 1,4-dioxan-2-yl, 1,3-dioxepan-2-yl, 1,3-dioxepan-4-yl, 1,3-dioxepan-5-yl, 1,4-dioxepan-2-yl, 1,4-dioxepan-5-yl and 1,4-dioxepan-6-yl.

In the case of a doubly substituted amino group, such as dialkylamino, these two substituents may be the same or different.

Halogen is fluorine, chlorine, bromine or iodine. Haloalkyl, -alkenyl and -alkynyl are alkyl, alkenyl or alkynyl, respectively, substituted fully or partly by halogen, preferably by fluorine, chlorine and/or bromine, in particular by fluorine or chlorine, examples being CF₃, CHF₂, CH₂F, CF₃CF₂, CH₂FCHCl, CCl₃, CHCl₂, CH₂CH₂Cl, CH═CHCl, CH═CCl₂, C≡CCH₂Cl; haloalkoxy is, for example, OCF₃, OCHF₂, OCH₂F, CF₃CF₂O, OCH₂CF₃ and OCH₂CH₂Cl; similar comments apply to haloalkenyl and other halogen-substituted radicals.

Where a group is multiply substituted, this means that with regard to the combination of the various substituents it is necessary for the general principles of the construction of chemical compounds to be observed; in other words, such that no compounds are formed that are known to the skilled worker to be chemically unstable or not possible.

Where R⁸ is hydrogen the compounds of the formula (I) according to the invention may occur in different tautomeric structures as a function of external conditions, such as solvent and pH:

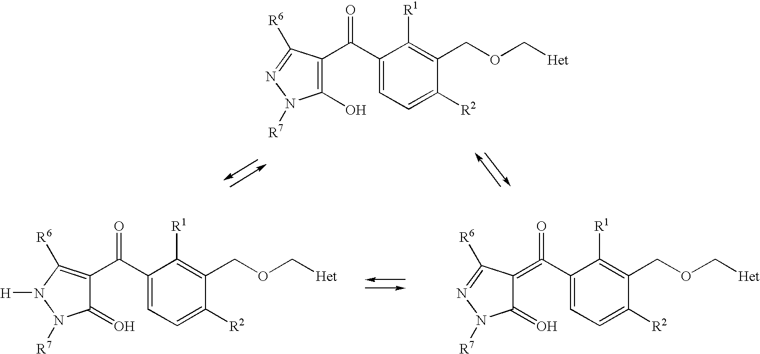

Tautomeric forms of the compounds according to the invention are also possible when R⁷ is hydrogen.

Depending on the identity and linking of the substituents, the compounds of the formula (I) may be in the form of stereoisomers. Where, for example, there are one or more asymmetric carbon atoms, then enantiomers and diastereomers may occur. Stereoisomers can be obtained from the as-prepared mixtures by standard separation methods, such as by chromatographic separation methods, for example. Stereoisomers can also be prepared selectively by using stereoselective reactions and employing optically active starting materials and/or auxiliaries. The invention also provides all stereoisomers and mixtures thereof that, while embraced by the formula (I), are not defined specifically.

Compounds of the formula (I) which have shown themselves to be advantageous include those in which R¹ and R² independently of one another are hydrogen, nitro, halogen, (C₁-C₄)-alkyl, (C₁-C₄)-haloalkyl, (C₂-C₆)-alkenyl, (C₂-C₆)-haloalkenyl, (C₂-C₆)-alkynyl, (C₂-C₆)-haloalkynyl, (C₃-C₆)-cycloalkyl, —OR⁴,S(O)ₙR⁴, SO₂OR⁴, SO₂N(R⁴)₂, NR⁴SO₂R⁴ or (C₁-C₆)-alkyl-S(O)ₙR⁴;

R⁴ is hydrogen, (C₁-C₄)-alkyl, (C₂-C₄)-alkenyl, (C₂-C₄)-alkynyl, (C₃-C₆)-cycloalkyl, phenyl or phenyl-(C₁-C₄)-alkyl, the six last-mentioned radicals being substituted by s radicals from the group consisting of cyano, nitro, R³, OR³, SR³ and N(R³)₂, and the other substituents and indices each have the definitions specified earlier on above.

Preferred compounds of the formula (I) are those in which
R³ is hydrogen or methyl;
R⁵ is cyano, nitro, halogen, (C₁-C₄)-alkoxycarbonyl, (C₁-C₄)-alkylcarbonyl, (C₁-C₄)-alkylcarbonyloxy, (C₁-C₄)-alkyl, (C₁-C₄)-haloalkyl, (C₁-C₄)-alkylthio, (C₁-C₄)-haloalkylthio, (C₁-C₆)-alkoxy or (C₁-C₆)-haloalkoxy, or R⁵ together with the carbon atom to which it is attached forms a carbonyl group, or two R⁵s together with the carbon atom to which they are attached form a 5-6-membered spiro ring, and the other substituents and indices each have the definitions specified earlier on above.

Particularly preferred compounds of the formula (I) are those in which
R⁵ is methyl, methoxy, ethyl, hexyl or chloromethyl, or R⁵ together with the carbon atom to which it is attached forms a carbonyl group, or two R⁵s together with the carbon atom to which they are attached form a 5-6-membered spiro ring;
R⁶ and R⁷ independently of one another are hydrogen, (C₁-C₄)-alkyl or cyclopropyl;
R⁸ is hydrogen, (C₁-C₄)-alkyl, (C₁-C₄)-haloalkyl, (C₁-C₄)-alkylcarbonyl, (C₁-C₄)-haloalkylcarbonyl, (C₁-C₄)-alkoxycarbonyl, (C₁-C₄)-alkylsulfonyl, (C₁-C₄)-haloalkylsulfonyl, phenylcarbonyl, phenylcarbonylmethyl, phenyloxycarbonyl or phenylsulfonyl, the phenyl ring of the four last-mentioned radicals being substituted by s radicals from the group consisting of halogen, nitro, cyano, (C₁-C₄)-alkyl, (C₁-C₄)-haloalkyl, (C₁-C₄)-alkoxy and (C₁-C₄)-haloalkoxy, and the other substituents and indices each have the definitions specified earlier on above.

Especially preferred compounds of the formula (I) are those in which
R¹ is chlorine, bromine, iodine, nitro, methyl or thiomethyl;
R² is chlorine, methylsulfonyl or ethylsulfonyl;
R⁶ and R⁷ independently of one another are hydrogen, methyl or ethyl, or cyclopropyl;
R⁸ is hydrogen, and the other substituents and indices each have the definitions specified earlier on above.

In all formulae below, the substituents and symbols, unless otherwise defined, have the same definition as that described under formula (I).

Compounds of the invention in which R⁸ is hydrogen can be prepared, for example, by the method indicated in scheme 1. For that purpose a compound of the formula (IIIa) is reacted, either in the presence of water removers, such as DCC, or following conversion to its acid chloride, with a pyrazole of the formula (IV), under base catalysis, and finally is treated with a source of cyanide. These methods are described for example in EP-A 0 369 803.

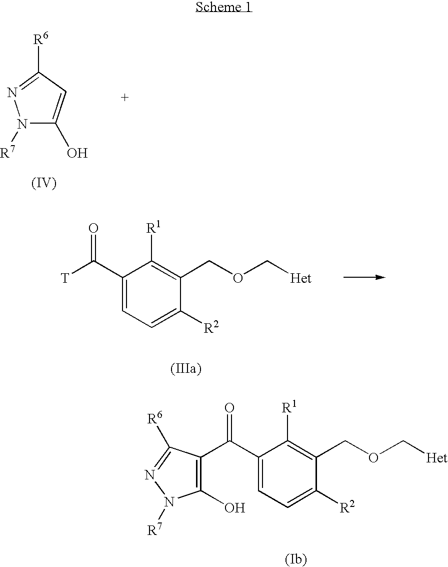

Compounds of the formula (I) according to the invention in which R⁸ is a radical other than hydrogen can be prepared, for example, in accordance with scheme 2 by substitution reactions that are known per se to the skilled worker. For that purpose compounds of the formula (Ib) are reacted with compounds of the formula (V) in which E is a leaving group capable of nucleophilic substitution. Such methods are known, for example, from WO 99/10328.

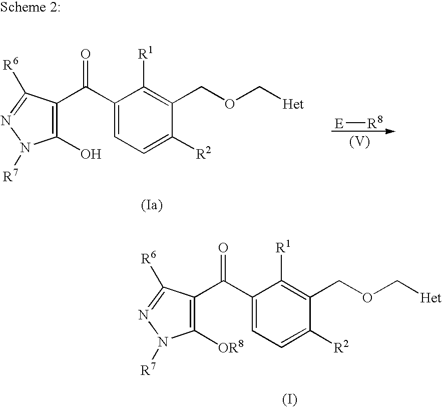

Compounds of the formula (IIIa) in which T is OH can be prepared, for example, in accordance with scheme 3 from compounds of the formula (VIa) in which Hal is halogen and a compound of the formula (VIIa).

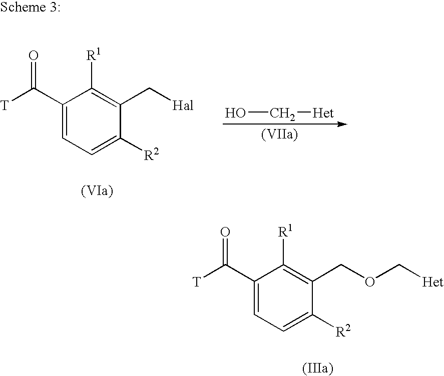

Compounds of the formula (IIIa) are also obtainable by reactions in accordance with scheme 4.

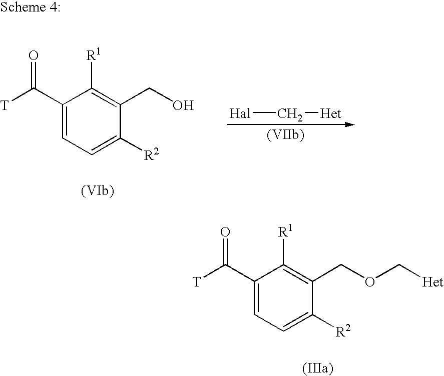

Compounds of the formula (VIa) and (VIb) are known from the literature or can be prepared by known methods, as described for example in WO 96/26200 and in German patent application 10144412.5, possessing an earlier priority but unpublished at the priority date of the present specification. Compounds of the formulae (VIIa) and (VIIb) are known to the skilled worker or can be prepared by reactions known to the skilled worker.

The compounds of the formula (I) according to the invention have an excellent herbicidal activity against a broad spectrum of economically important monocotyledonous and dicotyledonous weed plants. The active substances provide effective control even of perennial weeds which produce shoots from rhizomes, root stocks or other perennial organs and which cannot be easily controlled. In this context, it generally does not matter whether the substances are applied before sowing, pre-emergence or post-emergence. Some representatives of the monocotyledonous and dicotyledonous weed flora which can be controlled by the compounds according to the invention may be mentioned individually as examples, but this is not to be taken to mean a restriction to certain species. The monocotyledonous weed species which are controlled well are, for example, Avena, Lolium, Alopecurus, Phalaris, Echinochloa, Digitaria, Setaria and Cyperus species from the annual group, and Agropyron, Cynodon, Imperata and Sorghum or else perennial Cyperus species amongst the perennial species. In the case of dicotyledonous weed species, the spectrum of action extends to species such as, for example, Galium, Viola, Veronica, Lamium, Stellaria, Amaranthus, Sinapis, Ipomoea, Sida, Matricaria and Abutilon from the annual group, and Convolvulus, Cirsium, Rumex and Artemisia among the perennial weeds. Weed plants which are found under the specific culture conditions of rice, such as, for example, Echinochloa, Sagittaria, Alisma, Eleocharis, Scirpus and Cyperus, are also controlled outstandingly well by the active substances according to the invention. If the compounds according to the invention are applied to the soil surface prior to germination, then either emergence of the weed seedlings is prevented completely, or the weeds grow until they have reached the cotyledon stage but their growth then comes to a standstill and, after a period of three to four weeks, the plants eventually die completely. When the active substances are applied post-emergence to the green parts of the plants, growth also stops drastically very soon after the treatment, and the weeds remain at the growth stage of the time of application, or, after a certain period of time, they die completely so that in this way competition by the weeds, which is detrimental for the crop plants, is thus eliminated at a very early stage and in a sustained manner. In particular, the compounds according to the invention have an outstanding action against Amaranthus retroflexus, Avena sp., Echinochloa sp., Cyperus serotinus, Lolium multiflorum, Setaria viridis, Sagittaria pygmaea, Scirpus juncoides, Sinapis sp. and Stellaria media.

The compounds according to the invention have an outstanding herbicidal activity against monocotyledonous and dicotyledonous weeds, and yet crop plants of economically important crops such as, for example, wheat, barley, rye, rice, maize, sugar beet, cotton and soya suffer only negligible damage, if any. In particular, they are outstandingly well tolerated in wheat, maize and rice. This is why the present compounds are highly suitable for the selective control of unwanted vegetation in stands of agricultural useful plants or of ornamentals.

Owing to their herbicidal properties, the active substances can also be employed for controlling weed plants in crops of genetically modified plants which are known or are yet to be developed. As a rule, the transgenic plants are distinguished by particular advantageous properties, for example by resistances to certain pesticides, especially certain herbicides, by resistances to plant diseases or causative organisms of plant diseases, such as certain insects or microorganisms such as fungi, bacteria or viruses. Other particular properties concern for example the harvested material with regard to quantity, quality, storage life, composition and specific constituents. Thus, transgenic plants are known which have an increased starch content or whose starch quality has been modified, or those whose fatty acid composition in the harvested material is different.

The compounds of the formula (I) according to the invention or their salts are preferably employed in economically important transgenic crops of useful plants and ornamentals, for example cereals such as wheat, barley, rye, oats, millet, rice, cassava and maize, or else crops of sugar beet, cotton, soya, oilseed rape, potato, tomato, pea and other vegetables. The compounds of the formula (I) can preferably be employed as herbicides in crops of useful plants which are resistant, or have been genetically modified to be resistant, to the phytotoxic effects of the herbicides.

Conventional routes for the generation of novel plants which have modified properties compared with existing plants are, for example, traditional breeding methods and the generation of mutants. Alternatively, novel plants with modified properties can be generated with the aid of recombinant methods (see, for example, EP-A-0221044, EP-A-0131624). For example, several cases of the following have been described:
recombinant modifications of crop plants for the purposes of modifying the starch synthesized in the plants (e.g., WO 92/11376, WO 92/14827, WO 91/19806),
transgenic crop plants which exhibit resistances to certain herbicides of the glufosinate type (cf., e.g., EP-A-0242236, EP-A-242246), glyphosate type (WO 92/00377) or of the sulfonylurea type (EP-A-0257993, U.S. Pat. No. 5,013,659)
transgenic crop plants, for example cotton, with the ability to produce Bacillus thuringiensis toxins (Bt toxins), which make the plants resistant to certain pests (EP-A-0142924, EP-A-0193259),
transgenic crop plants with a modified fatty acid composition (WO 91/13972),

A large number of techniques in molecular biology, with the aid of which novel transgenic plants with modified properties can be generated, are known in principle; see, for example, Sambrook et al., 1989, Molecular Cloning, A Laboratory Manual, 2nd Ed., Cold Spring Harbor Laboratory Press, Cold Spring Harbor, N.Y.; or Winnacker “Gene und Klone” [Genes and Clones], VCH Weinheim 2nd Edition 1996 or Christou, “Trends in Plant Science” 1 (1996) 423-431.

To carry out such recombinant manipulations, nucleic acid molecules can be introduced into plasmids which permit a mutagenesis or a sequence alteration by recombination of DNA sequences. With the aid of the abovementioned standard processes, it is possible, for example, to carry out base substitutions, to remove part sequences or to add natural or synthetic sequences. The fragments can be provided with adapters or linkers to link the DNA fragments to each other.

Plant cells with a reduced activity of a gene product can be obtained, for example, by expressing at least one corresponding antisense RNA, a sense RNA for achieving a cosuppression effect, or the expression of at least one suitably constructed ribozyme which specifically cleaves transcripts of the abovementioned gene product.

To this end, it is possible, on the one hand, to use DNA molecules which encompass all of the coding sequence of a gene product including any flanking sequences which may be present, but also DNA molecules which only encompass portions of the coding sequence, it being necessary for these portions to be so long as to cause an antisense effect in the cells. Another possibility is the use of DNA sequences which have a high degree of homology with the coding sequences of a gene product, but are not completely identical.

When expressing nucleic acid molecules in plants, the protein synthesized may be localized in any desired compartment of the plant cell. However, to achieve localization in a particular compartment, the coding region can, for example, be linked to DNA sequences which ensure localization in a particular compartment. Such sequences are known to the skilled worker (see, for example, Braun et al., EMBO J. 11 (1992), 3219-3227; Wolter et al., Proc. Natl. Acad. Sci. USA 85 (1988), 846-850; Sonnewald et al., Plant J. 1 (1991), 95-106).

The transgenic plant cells can be regenerated by known techniques to give intact plants. In principle, the transgenic plants can be plants of any desired plant species, i.e., both monocotyledonous and dicotyledonous plants.

Thus, transgenic plants can be obtained which exhibit modified properties owing to the overexpression, suppression or inhibition of homologous (i.e., natural) genes or gene sequences or expression of heterologous (i.e., foreign) genes or gene sequences.

When using the active substances according to the invention in transgenic crops, effects are frequently observed—in addition to the effects against weed plants to be observed in other crops—which are specific for the application in the transgenic crop in question, for example a modified or specifically widened controllable weed spectrum, modified application rates which may be employed for the application, preferably good combining ability with the herbicides to which the transgenic crop is resistant, and an effect on the growth and yield of the transgenic crop plants. The invention therefore also relates to the use of the compounds according to the invention as herbicides for controlling weed plants in transgenic crop plants.

The substances according to the invention additionally have outstanding growth-regulatory properties in crop plants. They engage in the plants' metabolism in a regulatory fashion and can thus be employed for the targeted influencing of plant constituents and for facilitating harvesting, such as, for example, by triggering desiccation and stunted growth. Moreover, they are also suitable for generally controlling and inhibiting unwanted vegetative growth without destroying the plants in the process. Inhibiting the vegetative growth plays an important role in many monocotyledonous and dicotyledonous crops, allowing lodging to be reduced or prevented completely.

The compounds according to the invention can be employed in the form of wettable powders, emulsifiable concentrates, sprayable solutions, dusts or granules in the customary preparations. The invention therefore further relates to herbicidal compositions comprising compounds of the formula (I). The compounds of the formula (I) can be formulated in various ways, depending on the prevailing biological and/or chemico-physical parameters. Examples of suitable formulations which are possible are: wettable powders (WP), water-soluble powders (SP), water-soluble concentrates, emulsifiable concentrates (EC), emulsions (EW), such as oil-in-water and water-in-oil emulsions, sprayable solutions, suspension concentrates (SC), oil- or water-based dispersions, oil-miscible solutions, capsule suspensions (CS), dusts (DP), seed-dressing products, granules for spreading and soil application, granules (GR) in the form of microgranules, spray granules, coated granules and adsorption granules, water-dispersible granules (WG), water-soluble granules (SG), ULV formulations, microcapsules and waxes. These individual formulation types are known in principle and are described, for example, in Winnacker-Küchler, “Chemische Technologie” [Chemical Technology], Volume 7, C. Hauser Verlag Munich, 4th Ed. 1986, Wade van Valkenburg, “Pesticide Formulations”, Marcel Dekker, N.Y., 1973; K. Martens, “Spray Drying” Handbook, 3rd Ed. 1979, G. Goodwin Ltd. London.

The formulation auxiliaries required, such as inert materials, surfactants, solvents and further additives, are likewise known and are described, for example, in: Watkins, “Handbook of Insecticide Dust Diluents and Carriers”, 2nd Ed., Darland Books, Caldwell N.J., H.v. Olphen, “Introduction to Clay Colloid Chemistry”; 2nd Ed., J. Wiley & Sons, N.Y.; C. Marsden, “Solvents Guide”; 2nd Ed., Interscience, N.Y. 1963; McCutcheon's “Detergents and Emulsifiers Annual”, MC PubI. Corp., Ridgewood N.J.; Sisley and Wood, “Encyclopedia of Surface Active Agents”, Chem. Publ. Co. Inc., N.Y. 1964; Schönfeldt, “Grenzflächenaktive Äthylenoxidaddukte” [Surface-active ethylene oxide adducts], Wiss. Verlagsgesell., Stuttgart 1976; Winnacker-Küchler, “Chemische Technologie”, Volume 7, C. Hauser Verlag Munich, 4th Ed. 1986.

Wettable powders are preparations which are uniformly dispersible in water and which, in addition to the active substance, also contain ionic and/or nonionic surfactants (wetters, dispersants), for example polyoxyethylated alkylphenols, polyoxyethylated fatty alcohols, polyoxyethylated fatty amines, fatty alcohol polyglycol ether sulfates, alkanesulfonates, alkyIbenzenesulfonates, sodium 2,2′-dinaphthylmethane-6,6′-disulfonate, sodium lignosulfonate, sodium dibutylnaphthalenesulfonate or else sodium oleoylmethyltaurate, in addition to a diluent or inert substance. To prepare the wettable powders, the herbicidal active substances are ground finely, for example in customary equipment such as hammer mills, blowing mills and air-jet mills, and simultaneously or subsequently mixed with the formulation auxiliaries.

Emulsifiable concentrates are prepared by dissolving the active substance in an organic solvent, such as butanol, cyclohexanone, dimethylformamide, xylene or else higher-boiling aromatics or hydrocarbons or mixtures of the organic solvents with addition of one or more ionic and/or nonionic surfactants (emulsifiers). Examples of emulsifiers which can be used are: calcium alkylarylsulfonate salts such as calcium dodecylbenzenesulfonate, or nonionic emulsifiers such as fatty acid polyglycol esters, alkylaryl polyglycol ethers, fatty alcohol polyglycol ethers, propylene oxide/ethylene oxide condensates, alkyl polyethers, sorbitan esters such as, for example, sorbitan fatty acid esters or polyoxyethylene sorbitan esters such as, for example, polyoxyethylene sorbitan fatty acid esters.

Dusts are obtained by grinding the active substance with finely divided solid materials, for example talc, natural clays such as kaolin, bentonite and pyrophyllite, or diatomaceous earth.

Suspension concentrates can be water-based or oil-based. They can be prepared for example by wet-grinding by means of customary bead mills, if appropriate with addition of surfactants, as have already been mentioned for example above in the case of the other formulation types.

Emulsions, for example oil-in-water emulsions (EW), can be prepared for example by means of stirrers, colloid mills and/or static mixers using aqueous organic solvents and, if appropriate, surfactants as have already been mentioned for example above in the case of the other formulation types.

Granules can be prepared either by spraying the active substance onto adsorptive, granulated inert material or by applying active substance concentrates to the surface of carriers such as sand, kaolinites or granulated inert material with the aid of tackifiers, for example polyvinyl alcohol, sodium polyacrylate or else mineral oils. Suitable active substances can also be granulated in the fashion which is conventional for the production of fertilizer granules, if desired as a mixture with fertilizers.

Water-dispersible granules are generally prepared by customary methods such as spray drying, fluidized-bed granulation, disk granulation, mixing with high-speed mixers and extrusion without solid inert material.

To prepare disk granules, fluidized-bed granules, extruder granules and spray granules, see, for example, processes in “Spray-Drying Handbook” 3rd ed. 1979, G. Goodwin Ltd., London; J. E. Browning, “Agglomeration”, Chemical and Engineering 1967, pages 147 et seq.; “Perry's Chemical Engineer's Handbook”, 5th Ed., McGraw-Hill, New York 1973, pp. 8-57.

For further details on the formulation of crop protection products see, for example, G. C. Klingman, “Weed Control as a Science”, John Wiley and Sons, Inc., New York, 1961, pages 81-96 and J. D. Freyer, S. A. Evans, “Weed Control Handbook”, 5th Ed., Blackwell Scientific Publications, Oxford, 1968, pages 101-103.

As a rule, the agrochemical preparations comprise 0.1 to 99% by weight, in particular 0.1 to 95% by weight, of active substance of the formula (I). In wettable powders, the active substance concentration is, for example, approximately 10 to 90% by weight, the remainder to 100% by weight being composed of customary formulation constituents. In the case of emulsifiable concentrates, the active substance concentration can amount to approximately 1 to 90, preferably 5 to 80% by weight. Formulations in the form of dusts comprise 1 to 30% by weight of active substance, preferably in most cases 5 to 20% by weight of active substance, and sprayable solutions comprise approximately 0.05 to 80, preferably 2 to 50% by weight of active substance. In the case of water-dispersible granules, the active substance content depends partly on whether the active compound is in liquid or solid form and on the granulation auxiliaries, fillers and the like which are being used. In the case of the water-dispersible granules, for example, the active substance content is between 1 and 95% by weight, preferably between 10 and 80% by weight.

In addition, the active substance formulations mentioned comprise, if appropriate, the stickers, wetters, dispersants, emulsifiers, penetrants, preservatives, antifreeze agents, solvents, fillers, carriers, colorants, antifoams, evaporation inhibitors, and pH and viscosity regulators which are conventional in each case.

Based on these formulations, it is also possible to prepare combinations with other pesticidally active substances such as, for example, insecticides, acaricides, herbicides, fungicides, and with safeners, fertilizers and/or growth regulators, for example in the form of a readymix or a tank mix.

Active substances which can be employed in combination with the active substances according to the invention in mixed formulations or in the tank mix are, for example, known active substances as are described, for example, in Weed Research 26, 441-445 (1986) or “The Pesticide Manual”, 11th edition, The British Crop Protection Council and the Royal Soc. of Chemistry, 1997 and literature cited therein. Known herbicides which are to be mentioned, and can be combined with the compounds of the formula (I), are, for example, the following active substances (note: the compounds are either designated by the common name according to the International Organization for Standardization (ISO) or using the chemical name, if appropriate together with a customary code number): acetochlor; acifluorfen; aclonifen; AKH 7088, i.e., [[[1-[5-[2-chloro-4-(trifluoromethyl)-phenoxy]-2-nitrophenyl]-2-methoxyethylidene]amino]oxy]acetic acid and its methyl ester; alachlor; alloxydim; ametryn; amidosulfuron; amitrol; AMS, i.e., ammonium sulfamate; anilofos; asulam; atrazine; azimsulfurone (DPX-A8947); aziprotryn; barban; BAS 516 H, i.e. 5-fluoro-2-phenyl-4H-3,1-benzoxazin-4-one; benazolin; benfluralin; benfuresate; bensulfuron-methyl; bensulide; bentazone; benzofenap; benzofluor; benzoylprop-ethyl; benzthiazuron; bialaphos; bifenox; bromacil; bromobutide; bromofenoxim; bromoxynil; bromuron; buminafos; busoxinone; butachlor; butamifos; butenachlor; buthidazole; butralin; butylate; cafenstrole (CH-900); carbetamide; cafentrazone (ICI-A0051); CDM, i.e. 2-chloro-N,N-di-2-propenylacetamide; CDEC, i.e., 2-chloroallyl diethyidithiocarbamate; chlomethoxyfen; chloramben; chlorazifop-butyl, chlormesulon (ICI-A0051); chlorbromuron; chlorbufam; chlorfenac; chlorflurecol-methyl; chloridazon; chlorimuron ethyl; chlornitrofen; chlorotoluron; chloroxuron; chlorpropham; chlorsulfuron; chlorthal-dimethyl; chlorthiamid; cinmethylin; cinosulfuron; clethodim; clodinafop and its ester derivatives (for example clodinafop-propargyl); clomazone; clomeprop; cloproxydim; clopyralid; cumyluron (JC 940); cyanazine; cycloate; cyclosulfamuron (AC 104); cycloxydim; cycluron; cyhalofop and its ester derivatives (for example butyl ester, DEH-1 12); cyperquat; cyprazine; cyprazole; daimuron; 2,4-DB; dalapon; desmedipham; desmetryn; di-allate; dicamba; dichlobenil; dichlorprop; diclofop and its esters such as diclofop-methyl; diethatyl; difenoxuron; difenzoquat; diflufenican; dimefuron; dimethachlor; dimethametryn; dimethenamid (SAN-582H); dimethazone, clomazon; dimethipin; dimetrasulfuron, dinitramine; dinoseb; dinoterb; diphenamid; dipropetryn; diquat; dithiopyr; diuron; DNOC; eglinazine-ethyl; EL 77, i.e., 5-cyano-1-(1,1-dimethylethyl)-N-methyl-1H-pyrazole-4-carboxamide; endothal; EPTC; esprocarb; ethalfluralin; ethametsulfuron-methyl; ethidimuron; ethiozin; ethofumesate; F5231, i.e., N-[2-chloro-4-fluoro-5-[4-(3-fluoropropyl)4,5-dihydro-5-oxo-1H-tetrazol-1-yl]phenyl]ethanesulfonamide; ethoxyfen and its esters (for example ethylester, HN-252); etobenzanid (HW 52); fenoprop; fenoxan, fenoxaprop and fenoxaprop-P and their esters, for example fenoxaprop-P-ethyl and fenoxaprop-ethyl; fenoxydim; fenuron; flamprop-methyl; flazasulfuron; fluazifop and fluazifop-P and their esters, for example fluazifop-butyl and fluazifop-P-butyl; fluchloralin; flumetsulam; flumeturon; flumiclorac and its esters (for example pentylester, S-23031); flumioxazin (S482); flumipropyn; flupoxam (KNW-739); fluorodifen; fluoroglycofen-ethyl; flupropacil (UBIC-4243); fluridone; flurochloridone; fluroxypyr; flurtamone; fomesafen; fosamine; furyloxyfen; glufosinate; glyphosate; halosafen; halosulfuron and its esters (for example methyl ester, NC-319); haloxyfop and its esters; haloxyfop-P (=R-haloxyfop) and its esters; hexazinone; imazapyr; imazamethabenz-methyl; imazaquin and salts such as the ammonium salt; ioxynil; imazethamethapyr; imazethapyr; imazosulfuron; isocarbamid; isopropalin; isoproturon; isouron; isoxaben; isoxapyrifop; karbutilate; lactofen; lenacil; linuron; MCPA; MCPB; mecoprop; mefenacet; mefluidid; mesotrione; metamitron; metazachlor; metham; methabenzthiazuron; methazole; methoxyphenone; methyidymron; metabenzuron, methobenzuron; metobromuron; metolachlor; metosulam (XRD 511); metoxuron; metribuzin; metsulfuron-methyl; MH; molinate; monalide; monolinuron; monuron; monocarbamide dihydrogensulfate; MT 128, i.e., 6-chloro-N-(3-chloro-2-propenyl)-5-methyl-N-phenyl-3-pyridazinamine; MT 5950, i.e., N-[3-chloro-4-(1-methylethyl)phenyl]-2-methylpentanamide; naproanilide; napropamide; naptalam; NC 310, i.e., 4-(2,4-dichlorobenzoyl)-1-methyl-5-benzyloxypyrazole; neburon; nicosulfuron; nipyraclophen; nitralin; nitrofen; nitrofluorfen; norflurazon; orbencarb; oryzalin; oxadiargyl (RP-020630); oxadiazon; oxyfluorfen; paraquat; pebulate; pendimethalin; perfluidone; phenisopham; phenmedipham; picloram; piperophos; piributicarb; pirifenop-butyl; pretilachlor; primisulfuron-methyl; procyazine; prodiamine; profluralin; proglinazine-ethyl; prometon; prometryn; propachlor; propanil; propaquizafop and its esters; propazine; propham; propisochlor; propyzamide; prosulfalin; prosulfocarb; prosulfuron (CGA-152005); prynachlor; pyrazolinate; pyrazon; pyrazosulfuron-ethyl; pyrazoxyfen; pyridate; pyrithiobac (KIH-2031); pyroxofop and its esters (for example propargyl ester); quinclorac; quinmerac; quinofop and its ester derivatives, quizalofop and quizalofop-P and their ester derivatives for example quizalofop-ethyl; quizalofop-P-tefuryl and -ethyl; renriduron; rimsulfuron (DPX-E 9636); S 275, i.e., 2-[4-chloro-2-fluoro-5-(2-propynyloxy)phenyl]-4,5,6,7-tetrahydro-2H-indazole; secbumeton; sethoxydim; siduron; simazine; simetryn; SN 106279, i.e., 2-[[7-[2-chloro-4-(trifluoromethyl)phenoxy]-2-naphthalenyl]oxy]propanoic acid and its methyl ester; suclotrione; sulfentrazon (FMC-97285, F-6285); sulfazuron; sulfometuron-methyl; sulfosate (ICI-A0224); TCA; tebutam (GCP-5544); tebuthiuron; terbacil; terbucarb; terbuchlor; terbumeton; terbuthylazine; terbutryn; TFH 450, i.e., N,N-diethyl-3-[(2-ethyl-6-methylphenyl)sulfonyl]-1H-1,2,4-triazole-1-carboxamide; thenylchlor (NSK-850); thiazafluron; thiazopyr (Mon-13200); thidiazimin (SN-24085); thiobencarb; thifensulfuron-methyl; tiocarbazil; tralkoxydim; tri-allate; triasulfuron; triazofenamide; tribenuron-methyl; triclopyr; tridiphane; trietazine; trifluralin; triflusulfuron and esters (for example methyl ester, DPX-66037); trimeturon; tsitodef; vernolate; WL 110547, i.e., 5-phenoxy-1-[3-(trifluoromethyl)phenyl]-1H-tetrazole; UBH-509; D-489; LS 82-556; KPP-300; NC-324; NC-330; KH-218; DPX-N8189; SC-0774; DOWCO-535; DK-8910; V-53482; PP-600; MBH-001; KIH-9201; ET-751; KIH-6127 and KIH-2023.

For use, the formulations, which are present in commercially available form, are diluted in the customary manner, for example using water in the case of wettable powders, emulsifiable concentrates, dispersions and water-dispersible granules. Preparations in the form of dusts, soil granules, granules for spreading and sprayable solutions are usually not diluted any further with other inert substances prior to use. The required application rate of the compounds of the formula (I) varies with the external conditions such as, inter alia, temperature, humidity and the nature of the herbicide used. It can vary within wide limits, for example between 0.001 and 1.0 kg/ha or more of active substance, but it is preferably between 0.005 and 750 g/ha.

The examples which follow illustrate the invention.

### A. CHEMICAL EXAMPLES

Preparation of 2-[2-chloro-3-({[2-(chloromethyl)-2-methyl-1,3-dioxolan-4-yl]methoxy}methyl)4-(methylsulfonyl)benzoyl]cyclohexane-1,3-dione (tabular example No. 1.1)

Step 1: 2-Chloro-3-({[2-(chloromethyl)-2-methyl-1,3-dioxolan-4-yl]methoxy}methyl)-4-(methylsulfonyl)benzoic Acid

5 ml of DMF and 3.58 g (22 mmol) of 2-(chloromethyl)-2-methyl-1,3-dioxolan-4-yl]methanol were introduced at RT and 0.85 g (21 mmol) of 60% NaH was added (strong evolution of gas). The mixture was left with stirring for one hour and 3 g (11 mmol) of 3-bromomethyl-2-chloro-4-methylsulfonylbenzoic acid were then added. Stirring was then continued for 1 hour. The mixture was diluted with 50 ml of water, acidified with KHSO₄ solution and extracted with chloroform. The organic phases were dried with MgSO₄, filtered and concentrated. This gave 3.568 g of viscous oil as a crude product, which was purified by chromatography. Yield: 1.28 g (3.5 mmol) 32% as a colorless oil with a purity of 91% by HPLC.

¹H-NMR: δ [CDCl₃] 1.45 and 1.5 (2s, 3H), 3.3 (s, 3H), 3.5 (m, 2H), 3.8 (m, 3H), 4.15 (m, 1H), 4.45 (m,1H), 5.25 (s, 2H), 7.98 (d,1H), 8.08 (d, 1H)

Step 2: 3-Oxocyclohex-1-en-1-yl 2-chloro-3-({[2-(chloromethyl)-2-methyl-1,3-dioxolan-4-yl]methoxy}methyl)4-(methylsulfonyl )benzoate

0.58 g of crude 2-chloro-3-({[2-(chloromethyl)-2-methyl-1,3-dioxolan-4-yl]methoxy}-methyl)-4-(methylsulfonyl)benzoic acid, 0.496 g (4 mmol) of cyclohexanedione and 0.364 g (2 mmol) of N-(3-dimethylaminopropyl)-N′-ethylcarbodiimide hydrochloride were dissolved in 5 ml of CH₂Cl₂ and the solution was stirred at RT for 4 h. It was then washed with water and NaHCO₃ solution, dried over MgSO₄, filtered with suction over silica gel and concentrated. Yield: 0.428 g (0.9 mmol) 52% as a yellow oil having a purity of 80% by HPLC.

¹H-NMR: δ [CDCl₃] 1.45 and 1.5 (2s, 3H), 2.15 (m, 2H), 2.5 (t, 2H), 2.7 (m, 2H), 3.3 (s, 3H), 3.5 (m, 2H), 3.8 (m, 3H), 4.15 (m, 1H), 4.45 (m, 1H), 5.25 (s, 2H), 6.1 (s, 1H), 7.9 (d,1H), 8.09 (d, 1H)

Step 3: 2-[2-Chloro-3-({[2-(chloromethyl)-2-methyl-1,3-dioxolan-4-yl]-methoxy}methyl)-4-(methylsulfonyl)benzoyl]cyclohexane-1,3-dione

0.385 g (1 mmol) of 3-oxocyclohex-1-en-1-yl 2-chloro-3-({[2-(chloromethyl)-2-methyl-1,3-dioxolan-4-yl]methoxy}methyl)-4-(methylsulfonyl)benzoate was dissolved in 5 ml of CH₃CN, and, with stirring, 0.123 g (1 mmol) of NEt₃, 0.017 g of KCN and 0.006 g of acetone cyanohydrin were added. The mixture was left with stirring at RT for 40 hours and concentrated. It was acidified with KHSO₄ solution and extracted with CH₂Cl₂. The organic solution was dried with MgSO₄, filtered and concentrated on a rotary evaporator. The crude product was purified by chromatography. Yield: 0.247 g (0.53 mmol) 77% colorless oil having a purity of 97% by HPLC.

The abbreviations used here have the following definitions:

| | | |
|---|---|---|
| cPr = cyclopropyl | nPr = n-propyl | nBu = n-butyl |
| Et = ethyl | Me = methyl | Ph = phenyl |
| RT = room temperature | | |

TABLE
Compounds of the formula (I) according to the invention in which the
substituents and symbols have the following definitions:
R⁶ = H R⁷ = Me R⁸ = H

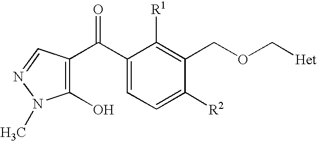

| No. | R¹ | R² | Het | ¹H-NMR |
|---|---|---|---|---|
| 1.1 | Cl | SO₂Me | 2-(Chloromethyl)-2-methyl-1,3-dioxolan-4-yl | ¹H-NMR: δ [CDCl₃] 1.45 and |
| | | | 1.5 (2s, 3H), 2.05 (m, 2H), 2.4 | |
| | | | (t, 2H), 2.8 (m, 2H), 3.3 (s, 3H), | |
| | | | 3.5 (m, 2H), 3.75 (m, 3H), 4.1 | |
| | | | (m, 1H), 4.4 (m, 1H), 5.2 | |
| | | | (s, 2H), 7.3 (d, 1H), 8.1 (d, 1H) | |
| 1.2 | Cl | SO₂Et | 2-(Chloromethyl)-2-methyl-1,3-dioxolan-4-yl | |
| 1.3 | Cl | Cl | 2-(Chloromethyl)-2-methyl-1,3-dioxolan-4-yl | |
| 1.4 | Br | SO₂Me | 2-(Chloromethyl)-2-methyl-1,3-dioxolan-4-yl | |
| 1.5 | Br | SO₂Et | 2-(Chloromethyl)-2-methyl-1,3-dioxolan-4-yl | |
| 1.6 | Br | Cl | 2-(Chloromethyl)-2-methyl-1,3-dioxolan-4-yl | |
| 1.7 | I | SO₂Me | 2-(Chloromethyl)-2-methyl-1,3-dioxolan-4-yl | |
| 1.8 | I | SO₂Et | 2-(Chloromethyl)-2-methyl-1,3-dioxolan-4-yl | |
| 1.9 | I | Cl | 2-(Chloromethyl)-2-methyl-1,3-dioxolan-4-yl | |
| 1.10 | Me | SO₂Me | 2-(Chloromethyl)-2-methyl-1,3-dioxolan-4-yl | |
| 1.11 | Me | SO₂Et | 2-(Chloromethyl)-2-methyl-1,3-dioxolan-4-yl | |
| 1.12 | Me | Cl | 2-(Chloromethyl)-2-methyl-1,3-dioxolan-4-yl | |
| 1.13 | SMe | SO₂Me | 2-(Chloromethyl)-2-methyl-1,3-dioxolan-4-yl | |
| 1.14 | SMe | SO₂Et | 2-(Chloromethyl)-2-methyl-1,3-dioxolan-4-yl | |
| 1.15 | SMe | Cl | 2-(Chloromethyl)-2-methyl-1,3-dioxolan-4-yl | |
| 1.16 | SO₂Me | SO₂Me | 2-(Chloromethyl)-2-methyl-1,3-dioxolan-4-yl | |
| 1.17 | SO₂Me | SO₂Et | 2-(Chloromethyl)-2-methyl-1,3-dioxolan-4-yl | |
| 1.18 | SO₂Me | Cl | 2-(Chloromethyl)-2-methyl-1,3-dioxolan-4-yl | |
| 1.19 | NO₂ | SO₂Me | 2-(Chloromethyl)-2-methyl-1,3-dioxolan-4-yl | |
| 1.20 | NO₂ | SO₂Et | 2-(Chloromethyl)-2-methyl-1,3-dioxolan-4-yl | |
| 1.21 | NO₂ | Cl | 2-(Chloromethyl)-2-methyl-1,3-dioxolan-4-yl | |
| 1.22 | Cl | SO₂Me | 2-Hexyl-1,3-dioxolan-4-yl | ¹H-NMR: δ [CDCl₃] 0.88 |
| | | | (m, 3H), 1.3 (m, 8H), 1.6 | |
| | | | (m, 2H), 3.3 and 3.45 (2s, 3H), | |
| | | | 3.7 (m), 3.85 (d), 4.1 (m), 4.3 | |
| | | | (d), 4.58 (t), 4.98 (t) (6H), 3.75 | |
| | | | (s, 3H), 5.25 (s, 2H), 7.35 | |
| | | | (s, 1H), 7.58 (d, 1H), 8.2 (d, 1H) | |
| 1.23 | Cl | SO₂Et | 2-Hexyl-1,3-dioxolan-4-yl | |
| 1.24 | Cl | Cl | 2-Hexyl-1,3-dioxolan-4-yl | |
| 1.25 | Br | SO₂Me | 2-Hexyl-1,3-dioxolan-4-yl | |
| 1.26 | Br | SO₂Et | 2-Hexyl-1,3-dioxolan-4-yl | |
| 1.27 | Br | Cl | 2-Hexyl-1,3-dioxolan-4-yl | |
| 1.28 | I | SO₂Me | 2-Hexyl-1,3-dioxolan-4-yl | |
| 1.29 | I | SO₂Et | 2-Hexyl-1,3-dioxolan-4-yl | |
| 1.30 | I | Cl | 2-Hexyl-1,3-dioxolan-4-yl | |
| 1.31 | Me | SO₂Me | 2-Hexyl-1,3-dioxolan-4-yl | |
| 1.32 | Me | SO₂Et | 2-Hexyl-1,3-dioxolan-4-yl | |
| 1.33 | Me | Cl | 2-Hexyl-1,3-dioxolan-4-yl | |
| 1.34 | SMe | SO₂Me | 2-Hexyl-1,3-dioxolan-4-yl | |
| 1.35 | SMe | SO₂Et | 2-Hexyl-1,3-dioxolan-4-yl | |
| 1.36 | SMe | Cl | 2-Hexyl-1,3-dioxolan-4 yl | |
| 1.37 | SO₂Me | SO₂Me | 2-Hexyl-1,3-dioxolan-4 yl | |
| 1.38 | SO₂Me | SO₂Et | 2-Hexyl-1,3-dioxolan-4-yl | |
| 1.39 | SO₂Me | Cl | 2-Hexyl-1,3-dioxolan-4-yl | |
| 1.40 | NO₂ | SO₂Me | 2-Hexyl-1,3-dioxolan-4-yl | |
| 1.41 | NO₂ | SO₂Et | 2-Hexyl-1,3-dioxolan-4-yl | |
| 1.42 | NO₂ | Cl | 2-Hexyl-1,3-dioxolan-4-yl | |
| 1.43 | Cl | SO₂Me | 1,4-Dioxan-2-yl | |
| 1.44 | Cl | SO₂Et | 1,4-Dioxan-2-yl | |
| 1.45 | Cl | Cl | 1,4-Dioxan-2-yl | |
| 1.46 | Br | SO₂Me | 1,4-Dioxan-2-yl | |
| 1.47 | Br | SO₂Et | 1,4-Dioxan-2-yl | |
| 1.48 | Br | Cl | 1,4-Dioxan-2-yl | |
| 1.49 | I | SO₂Me | 1,4-Dioxan-2-yl | |
| 1.50 | I | SO₂Et | 1,4-Dioxan-2-yl | |
| 1.51 | I | Cl | 1,4-Dioxan-2-yl | |
| 1.52 | Me | SO₂Me | 1,4-Dioxan-2-yl | |
| 1.53 | Me | SO₂Et | 1,4-Dioxan-2-yl | |
| 1.54 | Me | Cl | 1,4-Dioxan-2-yl | |
| 1.55 | SMe | SO₂Me | 1,4-Dioxan-2-yl | |
| 1.56 | SMe | SO₂Et | 1,4-Dioxan-2-yl | |
| 1.57 | SMe | Cl | 1,4-Dioxan-2-yl | |
| 1.58 | SO₂Me | SO₂Me | 1,4-Dioxan-2-yl | |
| 1.59 | SO₂Me | SO₂Et | 1,4-Dioxan-2-yl | |
| 1.60 | SO₂Me | Cl | 1,4-Dioxan-2-yl | |
| 1.61 | NO₂ | SO₂Me | 1,4-Dioxan-2-yl | |
| 1.62 | NO₂ | SO₂Et | 1,4-Dioxan-2-yl | |
| 1.63 | NO₂ | Cl | 1,4-Dioxan-2-yl | |
| 1.64 | Cl | SO₂Me | 5-Methyl-1,3-dioxan-5-yl | |
| 1.65 | Cl | SO₂Et | 5-Methyl-1,3-dioxan-5-yl | |
| 1.66 | Cl | Cl | 5-Methyl-1,3-dioxan-5-yl | ¹H-NMR: δ [CDCl₃] 0.85 |
| | | | (s, 3H), 3.4 (d, 2H), 3.6 (s, 2H), | |
| | | | 3.7 (s, 3H), 3.85 (d, 2H), 4.65 | |
| | | | (d, 1H), 4.85 (s, 1H), 4.9 | |
| | | | (d, 2H), 7.38 (d, 1H), 7.39 | |
| | | | (s, 1H), 7.42 (d, 1H), 8.7 (s, 1H) | |
| 1.67 | Br | SO₂Me | 5-Methyl-1,3-dioxan-5-yl | |
| 1.68 | Br | SO₂Et | 5-Methyl-1,3-dioxan-5-yl | |
| 1.69 | Br | Cl | 5-Methyl-1,3-dioxan-5-yl | |
| 1.70 | I | SO₂Me | 5-Methyl-1,3-dioxan-5-yl | |
| 1 71 | I | SO₂Et | 5-Methyl-1,3-dioxan-5-yl | |
| 1.72 | I | Cl | 5-Methyl-1,3-dioxan-5-yl | |
| 1.73 | Me | SO₂Me | 5-Methyl-1,3-dioxan-5-yl | |
| 1.74 | Me | SO₂Et | 5-Methyl-1,3-dioxan-5-yl | |
| 1.75 | Me | Cl | 5-Methyl-1,3-dioxan-5-yl | |
| 1.76 | SMe | SO₂Me | 5-Methyl-1,3-dioxan-5-yl | |
| 1.77 | SMe | SO₂Et | 5-Methyl-1,3-dioxan-5-yl | |
| 1.78 | SMe | Cl | 5-Methyl-1,3-dioxan-5-yl | |
| 1.79 | SO₂Me | SO₂Me | 5-Methyl-1,3-dioxan-5-yl | |
| 1.80 | SO₂Me | SO₂Et | 5-Methyl-1,3-dioxan-5-yl | |
| 1.81 | SO₂Me | Cl | 5-Methyl-1,3-dioxan-5-yl | |
| 1.82 | NO₂ | SO₂Me | 5-Methyl-1,3-dioxan-5-yl | |
| 1.83 | NO₂ | SO₂Et | 5-Methyl-1,3-dioxan-5-yl | |
| 1.84 | NO₂ | Cl | 5-Methyl-1,3-dioxan-5-yl | |
| 1.85 | Cl | SO₂Me | 5-Ethyl-2,2-dimethyl-1,3-dioxan-5-yl | |
| 1.86 | Cl | SO₂Et | 5-Ethyl-2,2-dimethyl-1,3-dioxan-5-yI | |
| 1.87 | Cl | Cl | 5-Ethyl-2,2-dimethyl-1,3-dioxan-5-yl | ¹H-NMR: δ [CDCl₃] 0.78 |
| | | | (t, 3H), 1.35 (q, 2H), 1.38 | |
| | | | (s, 3H), 1.4 (s, 3H), 3.58 | |
| | | | (d, 2H), 3.6 (s, 2H), 3.7 (d, 2H), | |
| | | | 3.72 (s, 3H), 4.85 (s, 2H), 7.35 | |
| | | | (d, 1H), 7.38 (s, 1H), 7.42 | |
| | | | (d, 1H), 8.7 (s, 1H) | |
| 1.88 | Br | SO₂Me | 5-Ethyl-2,2-dimethyl-1,3-dioxan-5-yl | |
| 1.89 | Br | SO₂Et | 5-Ethyl-2,2-dimethyl-1,3-dioxan-5-yl | |
| 1.90 | Br | Cl | 5-Ethyl-2,2-dimethyl-1,3-dioxan-5-yl | |
| 1.91 | I | SO₂Me | 5-Ethyl-2,2-dimethyl-1,3-dioxan-5-yl | |
| 1.92 | I | SO₂Et | 5-Ethyl-2,2-dimethyl-1,3-dioxan-5-yl | |
| 1.93 | I | Cl | 5-Ethyl-2,2-dimethyl-1,3-dioxan-5-yl | |
| 1.94 | Me | SO₂Me | 5-Ethyl-2,2-dimethyl-1,3-dioxan-5-yl | |
| 1.95 | Me | SO₂Et | 5-Ethyl-2,2-dimethyl-1,3-dioxan-5-yl | |
| 1.96 | Me | Cl | 5-Ethyl-2,2-dimethyl-1,3-dioxan-5-yl | |
| 1.97 | SMe | SO₂Me | 5-Ethyl-2,2-dimethyl-1,3-dioxan-5-yl | |
| 1.98 | SMe | SO₂Et | 5-Ethyl-2,2-dimethyl-1,3-dioxan-5-yl | |
| 1.99 | SMe | Cl | 5-Ethyl-2,2-dimethyl-1,3-dioxan-5-yl | |
| 1.100 | SO₂Me | SO₂Me | 5-Ethyl-2,2-dimethyl-1,3-dioxan-5-yl | |
| 1.101 | SO₂Me | SO₂Et | 5-Ethyl-2,2-dimethyl-1,3-dioxan-5-yl | |
| 1.102 | SO₂Me | Cl | 5-Ethyl-2,2-dimethyl-1,3-dioxan-5-yl | |
| 1.103 | NO₂ | SO₂Me | 5-Ethyl-2,2-dimethyl-1,3-dioxan-5-yl | |
| 1.104 | NO₂ | SO₂Et | 5-Ethyl-2,2-dimethyl-1,3-dioxan-5-yl | |
| 1.105 | NO₂ | Cl | 5-Ethyl-2,2-dimethyl-1,3-dioxan-5-yl | |
| 1.106 | Cl | SO_{2Me} | 5-Ethyl-1,3-dioxan-5-yl | |
| 1.107 | Cl | SO_{2Et} | 5-Ethyl-1,3-dioxan-5-yl | |
| 1.108 | Cl | Cl | 5-Ethyl-1,3-dioxan-5-yl | ¹H-NMR: δ [CDCl₃] 0.75 |
| | | | (t, 3H), 1.35 (q, 2H), 3.42 | |
| | | | (d, 2H), 3.62 (s, 2H), 3.72 | |
| | | | (s, 3H), 3.85 (d, 2H), 4.65 | |
| | | | (d, 1H), 4.85 (s, 1H), 4.92 | |
| | | | (d, 1H), 7.35 (d, 1H), 7.38 | |
| | | | (s, 1H), 7.45 (d, 1H), 8.95 | |
| | | | (s, 1H) | |
| 1.109 | Br | SO2Me | 5-Ethyl-1,3-dioxan-5-yl | |
| 1.110 | Br | SO2Et | 5-Ethyl-1,3-dioxan-5-yl | |
| 1.111 | Br | Cl | 5-Ethyl-1,3-dioxan-5-yl | |
| 1.112 | I | SO2Me | 5-Ethyl-1,3-dioxan-5-yl | |
| 1.113 | I | SO2Et | 5-Ethyl-1,3-dioxan-5-yl | |
| 1.114 | I | CI | 5-Ethyl-1,3-dioxan-5-yl | |
| 1.115 | Me | SO2Me | 5-Ethyl-1,3-dioxan-5-yl | |
| 1.116 | Me | SO2Et | 5-Ethyl-1,3-dioxan-5-yl | |
| 1.117 | Me | CI | 5-Ethyl-1,3-dioxan-5-yl | |
| 1.118 | SMe | SO2Me | 5-Ethyl-1,3-dioxan-5-yl | |
| 1.119 | SMe | SO2Et | 5-Ethyl-1,3-dioxan-5-yl | |
| 1.120 | SMe | Cl | 5-Ethyl-1,3-dioxan-5-yl | |
| 1.121 | SO₂Me | SO₂Me | 5-Ethyl-1,3-dioxan-5-yl | |
| 1.122 | SO₂Me | SO₂Et | 5-Ethyl-1,3-dioxan-5-yl | |
| 1.123 | SO₂Me | Cl | 5-Ethyl-1,3-dioxan-5-yl | |
| 1.124 | NO₂ | SO₂Me | 5-Ethyl-1,3-dioxan-5-yl | |
| 1.125 | NO₂ | SO₂Et | 5-Ethyl-1,3-dioxan-5-yl | |
| 1.126 | NO₂ | Cl | 5-Ethyl-1,3-dioxan-5-yl | |
| 1.127 | Cl | SO₂Me | 8-Methyl-6,10-dioxaspiro[4.5]dec-8-yl | |
| 1.128 | Cl | SO₂Et | 8-Methyl-6,10-dioxaspiro[4.5]dec-8-yl | |
| 1.129 | Cl | Cl | 8-Methyl-6,10-dioxaspiro[4.5]dec-8-yl | |
| 1.130 | Br | SO₂Me | 8-Methyl-6,10-dioxaspiro[4.5]dec-8-yl | |
| 1.131 | Br | SO₂Et | 8-Methyl-6,10-dioxaspiro[4.5]dec-8-yl | |
| 1.132 | Br | Cl | 8-Methyl-6,10-dioxaspiro[4.5]dec-8-yl | |
| 1.133 | I | SO₂Me | 8-Methyl-6,10-dioxaspiro[4.5]dec-8-yl | |
| 1.134 | I | SO₂Et | 8-Methyl-6,10-dioxaspiro[4.5]dec-8-yl | |
| 1.135 | I | Cl | 8-Methyl-6,10-dioxaspiro[4.5]dec-8-yl | |
| 1.136 | Me | SO₂Me | 8-Methyl-6,10-dioxaspiro[4.5]dec-8-yI | |
| 1.137 | Me | SO₂Et | 8-Methyl-6,10-dioxaspiro[4.5]dec-8-yl | |
| 1.138 | Me | Cl | 8-Methyl-6,10-dioxaspiro[4.5]dec-8-yl | |
| 1.139 | SMe | SO₂Me | 8-Methyl-6,10-dioxaspiro[4.5]dec-8-yl | |
| 1.140 | SMe | SO₂Et | 8-Methyl-6,10-dioxaspiro[4.5]dec-8-yl | |
| 1.141 | SMe | Cl | 8-Methyl-6,10-dioxaspiro[4.5]dec-8-yl | |
| 1.142 | SO₂Me | SO₂Me | 8-Methyl-6,10-dioxaspiro[4.5]dec-8-yl | |
| 1.143 | SO₂Me | SO₂Et | 8-Methyl-6,10-dioxaspiro[4.5]dec-8-yl | |
| 1.144 | SO₂Me | Cl | 8-Methyl-6,10-dioxaspiro[4.5]dec-8-yl | |
| 1.145 | NO₂ | SO₂Me | 8-Methyl-6,10-dioxaspiro[4.5]dec-8-yl | |
| 1.146 | NO₂ | SO₂Et | 8-Methyl-6,10-dioxaspiro[4.5]dec-8-yl | |
| 1.147 | NO₂ | Cl | 8-Methyl-6,10-dioxaspiro[4.5]dec-8-yl | |
| 1.148 | Cl | SO₂Me | 3-Methyl-1,5-dioxaspiro[5.5]undec-3-yl | ¹H.NMR δ [CDCl₃] 0.82 |
| | | | (s, 3H), 1.42 (m, 6H), 1.65 | |
| | | | (m, 2H), 1.82 (m, 2H), 3.22 | |
| | | | (s, 3H), 3.55 (d, 2H), 3.6 | |
| | | | (d, 2H), 3.7 (s, 2H), 3.72 | |
| | | | (s, 3H), 5.18 (s, 2H), 7.35 | |
| | | | (s, 1H), 7.58 (d, 1H), 8.19 | |
| | | | (d, 1H) | |
| 1.149 | Cl | SO₂Et | 3-Methyl-1,5-dioxaspiro[5.5]undec-3-yl | |
| 1.150 | Cl | Cl | 3-Methyl-1,5-dioxaspiro[5.5]undec-3-yl | ¹H-NMR: δ [CDCl₃] 0.82 |
| | | | (s, 3H), 1.4 (m, 2H), 1.5 | |
| | | | (m, 4H), 1.62 (m, 2H), 1.8 | |
| | | | (m, 2H), 3.52 (d, 2H), 3.55 | |
| | | | (s, 2H), 3.7 (d, 2H), 3.72 | |
| | | | (s, 3H), 4.85 (s, 2H), 7.35 | |
| | | | (d, 1H), 7.4 (s, 1H), 7.45 | |
| | | | (d, 1H), 9.7 (s, 1H) | |
| 1.151 | Br | SO₂Me | 3-Methyl-1,5-dioxaspiro[5.5]undec-3-yl | |
| 1.152 | Br | SO₂Et | 3-Methyl-1,5-dioxaspiro[5.5]undec-3-yl | |
| 1.153 | Br | Cl | 3-Methyl-1,5-dioxaspiro[5.5]undec-3-yl | |
| 1.154 | I | SO₂Me | 3-Methyl-1,5-dioxaspiro[5.5]undec-3-yl | |
| 1.155 | I | SO₂Et | 3-Methyl-1,5-dioxaspiro[5.5]undec-3-yl | |
| 1.156 | I | Cl | 3-Methyl-1,5-dioxaspiro[5.5]undec-3-yl | |
| 1.157 | Me | SO₂Me | 3-Methyl-1,5-dioxaspiro[5.5]undec-3-yl | |
| 1.158 | Me | SO₂Et | 3-Methyl-1,5-dioxaspiro[5.5]undec-3-yl | |
| 1.159 | Me | Cl | 3-Methyl-1,5-dioxaspiro[5.5]undec-3-yl | |
| 1.160 | SMe | SO2Me | 3-Methyl-1,5-dioxaspiro[5 5]undec-3-yl | |
| 1.161 | SMe | SO2Et | 3-Methyl-1,5-dioxaspiro[5.5]undec-3-yl | |
| 1.162 | SMe | CI | 3-Methyl-1,5-dioxaspiro[5.5]undec-3-yl | |
| 1.163 | SO₂Me | SO₂Me | 3-Methyl-1,5-dioxaspiro[5.5]undec-3-yl | |
| 1.164 | SO₂Me | SO₂Et | 3-Methyl-1,5-dioxaspiro[5.5]undec-3-yl | |
| 1.165 | SO₂Me | Cl | 3-Methyl-1,5-dioxaspiro[5.5]undec-3-yl | |
| 1.166 | NO₂ | SO₂Me | 3-Methyl-1,5-dioxaspiro[5.5]undec-3-yl | |
| 1.167 | NO₂ | SO₂Et | 3-Methyl-1,5-dioxaspiro[5.5]undec-3-yl | |
| 1.168 | NO₂ | Cl | 3-Methyl-1,5-dioxaspiro[5.5]undec-3-yl | |
| 1.169 | Cl | SO₂Me | 2,2,5-Trimethyl-1,3-dioxan-5-yl | |
| 1.170 | Cl | SO₂Et | 2,2,5-Trimethyl-1,3-dioxan-5-yl | |
| 1.171 | Cl | Cl | 2,2,5-Trimethyl-1,3-dioxan-5-yl | |
| 1.172 | Br | SO₂Me | 2,2,5-Trimethyl-1,3-dioxan-5-yl | |
| 1.173 | Br | SO₂Et | 2,2,5-Trimethyl-1,3-dioxan-5-yl | |
| 1.174 | Br | Cl | 2,2,5-Trimethyl-1,3-dioxan-5-yl | |
| 1.175 | I | SO₂Me | 2,2,5-Trimethyl-1,3-dioxan-5-yl | |
| 1.176 | I | SO₂Et | 2,2,5-Trimethyl-1,3-dioxan-5-yl | |
| 1.177 | I | Cl | 2,2,5-Trimethyl-1,3-dioxan-5-yl | |
| 1.178 | Me | SO₂Me | 2,2,5-Trimethyl-1,3-dioxan-5-yl | |
| 1.179 | Me | SO₂Et | 2,2,5-Trimethyl-1,3-dioxan-5-yl | |
| 1.180 | Me | Cl | 2,2,5-Trimethyl-1,3-dioxan-5-yl | |
| 1.181 | SMe | SO₂Me | 2,2,5-Trimethyl-1,3-dioxan-5-yl | |
| 1.182 | SMe | SO₂Et | 2,2,5-Trimethyl-1,3-dioxan-5-yl | |
| 1.183 | SMe | Cl | 2,2,5-Trimethyl-1,3-dioxan-5-yl | |
| 1.184 | SO₂Me | SO₂Me | 2,2,5-Trimethyl-1,3-dioxan-5-yl | |
| 1.185 | SO₂Me | SO₂Et | 2,2,5-Trimethyl-1,3-dioxan-5-yl | |
| 1.186 | SO₂Me | Cl | 2,2,5-Trimethyl-1,3-dioxan-5-yl | |
| 1.187 | NO₂ | SO₂Me | 2,2,5-Trimethyl-1,3-dioxan-5-yl | |
| 1.188 | NO₂ | SO₂Et | 2,2,5-Trimethyl-1,3-dioxan-5-yl | |
| 1.189 | NO₂ | Cl | 2,2,5-Trimethyl-1,3-dioxan-5-yl | |

### B. FORMULATION EXAMPLES

1. Dust

A dust is obtained by mixing 10 parts by weight of a compound of the formula (I) and 90 parts by weight of talc as inert substance and comminuting the mixture in a hammer mill.

2. Dispersible Powder

A wettable powder which is readily dispersible in water is obtained by mixing 25 parts by weight of a compound of the formula (I), 64 parts by weight of kaolin-containing quartz as inert substance, 10 parts by weight of potassium ligninsulfonate and 1 part by weight of sodium oleoylmethyltauride as wetter and dispersant, and grinding the mixture in a pinned-disk mill.

3. Dispersion Concentrate

A dispersion concentrate which is readily dispersible in water is obtained by mixing 20 parts by weight of a compound of the formula (I), 6 parts by weight of alkylphenol polyglycol ether (®Triton X 207), 3 parts by weight of isotridecanol polyglycol ether (8 EO) and 71 parts by weight of paraffinic mineral oil (boiling range for example approx. 255 to above 277° C.), and grinding the mixture in a ball mill to a fineness of below 5 microns.

4. Emulsifiable Concentrate

An emulsifiable concentrate is obtained from 15 parts by weight of a compound of the formula (I), 75 parts by weight of cyclohexanone as solvent and 10 parts by weight of oxyethylated nonylphenol as emulsifier.

5. Water-Dispersible Granules

Water-dispersible granules are obtained by mixing 75 parts by weight of a compound of the formula (I),

10″ calcium ligninsulfonate,
5″ sodium lauryl sulfate,
3″ polyvinyl alcohol and
7″ of kaolin,
   grinding the mixture in a pinned-disk mill and granulating the powder in a fluidized bed by spraying on water as granulation liquid.
   Water-dispersible granules are also obtained by homogenizing and precomminuting, in a colloid mill,
   25 parts by weight of a compound of the formula (I),
5″ sodium 2,2′-dinaphthylmethane-6,6′-disulfonate,
2″ sodium oleoylmethyltauride,
1″ polyvinyl alcohol,
17″ calcium carbonate and
50″ water,
   subsequently grinding the mixture in a bead mill, and atomizing and drying the resulting suspension in a spray tower by means of a single-fluid nozzle.

C. Biological Examples

1. Pre-Emergence Weed Action

Seeds of mono- and dicotyledonous broadleaf weed plants are placed in sandy loam in cardboard pots and covered with soil. The compounds according to the invention, formulated as wettable powders or emulsifiable concentrates, are then applied, in the form of an aqueous suspension or emulsion, at various dosages, onto the surface of the covering soil, at an application rate of 600 to 800 l of water per ha (converted). Following treatment, the pots are placed in the greenhouse and maintained under good growth conditions for the broadleaf weeds. The visual scoring of the plant damage or emergence damage is made when the test plants have emerged, after an experimental period of 3 to 4 weeks, in comparison to untreated controls. After the test plants have been left to stand in the greenhouse for 3 to 4 weeks under optimal growth conditions, the effect of the compounds is scored. In this experiment the compounds of the invention have outstanding activity against a broad spectrum of economically important monocotyledonous and dicotyledonous weed plants.

2. Post-Emergence Herbicidal Action Against Weed Plants

Seeds of mono- and dicotyledonous weed plants are placed in sandy loam in cardboard pots, covered with soil and grown in the greenhouse under good growth conditions. Two to three weeks after sowing, the test plants are treated at the three-leaf stage. The compounds according to the invention, formulated as wettable powders or as emulsifiable concentrates, are sprayed at various dosages onto the surface of the green plant parts at an application rate of 600 to 800 l of water per ha (converted). After the test plants have been left to stand in the greenhouse for 3 to 4 weeks under optimal growth conditions, the effect of the compounds is scored. In this test the compounds according to the invention exhibit outstanding activity against a broad spectrum of economically important monocotyledonous and dicotyledonous weed plants. Thus, for example, the compounds of Nos. 1.6 and 1.108 according to the invention, at a dosage of 320 g/ha, exhibit an activity of at least 80% against the weed plant Stellaria media.

3. Crop Plant Tolerance

In further greenhouse experiments, seeds of barley and of monocotyledonous and dicotyledonous weed plants are placed in sandy loam, covered with soil and placed in the greenhouse until the plants have developed two to three true leaves. Then they are treated with the compounds of the formula (I) according to the invention, as described above in section 2. Four to five weeks after the application and after having been left to stand in the greenhouse, visual scoring reveals that the compounds according to the invention are outstandingly well tolerated by important crop plants, in particular wheat, maize and rice.

1. A compound of the formula (I) or salt thereof 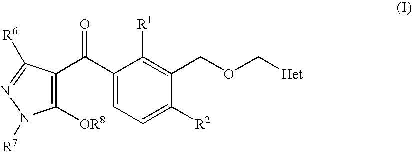
in which the radicals and indices have the following definitions:
R¹ and R² independently of one another are hydrogen, mercapto, nitro, halogen, cyano, thiocyanato, (C₁-C₆)-alkyl, (C₁-C₆)-haloalkyl, (C₂-C₆)-alkenyl, (C₂-C₆)-haloalkenyl, (C₂-C₆)-alkynyl, (C₃-C₆)-haloalkynyl, (C₃-C₆)-cycloalkyl, OR⁴, OCOR⁴, OSO₂R⁴, S(O)ₙR⁴, SO₂OR⁴, SO₂N(R⁴)₂, NR⁴SO₂R⁴, NR⁴COR⁴, (C₁-C₆)-alkyl-S(O)ₙR⁴, (C₁-C₆)-alkyl-OR⁴, (C₁-C₆)-alkyl-OCOR⁴, (C₁-C₆)-alkyl-OSO₂R⁴, (C₁-C₆)-alkyl-SO₂OR⁴, (C₁-C₆)-alkyl-SO₂N(R⁴)₂ or (C₁-C₆)-alkyl-NR⁴COR⁴;
R³ is hydrogen, (C₁-C₆)-alkyl, (C₂-C₆)-alkenyl or (C₂-C₆)-alkynyl;
R⁴ is hydrogen, (C₁-C₆)-alkyl, (C₂-C₆)-alkenyl, (C₂-C₆)-alkynyl, (C₃-C₆)-cycloalkyl, phenyl or phenyl-(C₁-C₆)-alkyl, the six last-mentioned radicals being substituted by s radicals from the group consisting of hydroxyl, mercapto, amino, cyano, nitro, thiocyanato, OR³, SR³, N(R³)₂, NOR³, OCOR³, SCOR³, NR³COR³, CO₂R³, COSR³, CON(R³)₂, (C₁-C₄)-alkyliminooxy, (C₁-C₄)-alkoxyamino, (C₁-C₄)-alkylcarbonyl, (C₁-C₄)-alkoxy-(C₂-C₆)-alkoxycarbonyl and (C₁-C₄)-alkylsulfonyl;
R⁶ and R⁷ independently of one another are hydrogen, (C₁-C₆)-alkyl, (C₁-C₆)-haloalkyl or (C₃-C₆)-cyclopropyl;
R⁸ is hydrogen, (C₁-C₆)-alkyl, (C₁-C₆)-haloalkyl, (C₁-C₆)-alkylcarbonyl, (C₁-C₆)-haloalkylcarbonyl, (C₁-C₆)-alkoxycarbonyl, (C₁-C₆)-alkylsulfonyl, (C₁-C₆)-haloalkylsulfonyl, phenylcarbonyl, phenylcarbonylmethyl, phenyloxycarbonyl or phenylsulfonyl, the phenyl ring of the four last-mentioned radicals being substituted by s radicals from the group consisting of halogen, nitro, cyano, (C₁-C₆)-alkyl, (C₁-C₆)-haloalkyl, (C₁-C₆)-alkoxy and (C₁-C₆)-haloalkoxy;
Het is a fully saturated heterocyclic group whose ring atoms are composed of 2 oxygen atoms and 2, 3, 4 or 5 carbon atoms;
and Het is substituted by n radicals R⁵;
n is 0, 1 or 2;
s is 0, 1, 2 or 3;
R⁵ is hydroxyl, mercapto, amino, cyano, nitro, halogen, formyl, (C₁-C₆)-alkylamino, (C₁-C₆)-dialkylamino, (C₁-C₆)-alkoxycarbonyl, (C₁-C₆)-alkylcarbonyl, (C₁-C₄)-alkylcarbonyloxy, (C₁-C₆)-alkyl, (C₁-C₆)-haloalkyl, (C₁-C₆)-alkylthio, (C₁-C₆)-haloalkylthio, (C₁-C₆)-alkoxy or (C₁-C₆)-haloalkoxy
or R⁵ together with the carbon atom to which it is attached forms a carbonyl group,
or two R⁵s together with the carbon atom to which they are attached form a 3- to 6-membered spiro ring.

2. A compound as claimed in claim 1 in which
R¹ and R² independently of one another are hydrogen, nitro, halogen, (C₁-C₄)-alkyl, (C₁-C₄)-haloalkyl, (C₂-C₆)-alkenyl, (C₂-C₆)-haloalkenyl, (C₂-C₆)-alkynyl, (C₂-C₆)-haloalkynyl, (C₃-C₆)-cycloalkyl, —OR⁴, S(O)ₙR⁴, SO₂OR⁴, SO₂N(R⁴)₂, NR⁴SO₂R⁴ or (C₁-C₆)-alkyl-S(O)ₙR⁴;
R⁴ is hydrogen, (C₁-C₄)-alkyl, (C₂-C₄)-alkenyl, (C₂-C₄)-alkynyl, (C₃-C₆)-cycloalkyl, phenyl or phenyl-(C₁-C₄)-alkyl, the six last-mentioned radicals being substituted by s radicals from the group consisting of cyano, nitro, R³, OR³, SR³ and N(R³)₂.

3. A compound as claimed in claim 1 in which
R³ is hydrogen or methyl;
R⁵ is cyano, nitro, halogen, (C₁-C₄)-alkoxycarbonyl, (C₁-C₄)-alkylcarbonyl, (C₁-C₄)-alkylcarbonyloxy, (C₁-C₄)-alkyl, (C₁-C₄)-haloalkyl, (C₁-C₄)-alkylthio, (C₁-C₄)-haloalkylthio, (C₁-C₆)-alkoxy or (C₁-C₆)-haloalkoxy,
or R⁵ together with the carbon atom to which it is attached forms a carbonyl group,
or two R⁵s together with the carbon atom to which they are attached form a 5-6-membered spiro ring.

4. A compound as claimed in claim 1, in which
R⁵ is methyl, methoxy, ethyl, hexyl or chloromethyl,
or R⁵ together with the carbon atom to which it is attached forms a carbonyl group,
or two R⁵s together with the carbon atom to which they are attached form a 5-6-membered spiro ring;
R⁶ and R⁷ independently of one another are hydrogen, (C₁-C₄)-alkyl or cyclopropyl;
R⁸ is hydrogen, (C₁-C₄)-alkyl, (C₁-C₄)-haloalkyl, (C₁-C₄)-alkylcarbonyl, (C₁-C₄)-haloalkylcarbonyl, (C₁-C₄)-alkoxycarbonyl, (C₁-C₄)-alkylsulfonyl, (C₁-C₄)-haloalkylsulfonyl, phenylcarbonyl, phenylcarbonylmethyl, phenyloxycarbonyl or phenylsulfonyl, the phenyl ring of the four last-mentioned radicals being substituted by s radicals from the group consisting of halogen, nitro, cyano, (C₁-C₄)-alkyl, (C₁-C₄)-haloalkyl, (C₁-C₄)-alkoxy and (C₁-C₄)-haloalkoxy.

5. A compound as claimed in claim 1 in which
R¹ is chlorine, bromine, iodine, nitro, methyl or thiomethyl;
R² is chlorine, methylsulfonyl or ethylsulfonyl;
R⁶ and R⁷ independently of one another are hydrogen, methyl or ethyl, or cyclopropyl;
R⁸ is hydrogen.

6. A herbicidal composition comprising a herbicidally active amount of at least one compound of the formula (I) as claimed in claim 1.

7. The herbicidal composition as claimed in claim 6 as a mixture with formulation auxiliaries.

8. A method of controlling unwanted plants which comprises applying to the plants or to the locus of unwanted plant growth an effective amount of at least one compound of the formula (I) as claimed in claim 1.

9. (canceled)

10. The method as claimed in claim 8, wherein the unwanted plants are located in crops of useful plants.

11. The method as claimed in claim 10, wherein the useful plants are transgenic plants.

12. A method of controlling unwanted plants which comprises applying to the plants or to the locus of unwanted plant growth an effective amount of a herbicidal composition as claimed in claim 6.
